(19) United States
(12) Patent Application Publication
(10) Pub. No.: US 2026/0076943 A1
(43) Pub. Date: Mar. 19, 2026

(54) METHOD FOR IMPROVING SKELETAL HEALTH

(71) Applicant: THE TRUSTEES OF INDIANA UNIVERSITY, Bloomington, IN (US)

(72) Inventors: Keith Gerard Avin, Carmel, IN (US); Sharon Moe, Indianapolis, IN (US); Michael L. Schulte, Carmel, IN (US)

(21) Appl. No.: 19/333,029

(22) Filed: Sep. 18, 2025

Related U.S. Application Data
(60) Provisional application No. 63/696,443, filed on Sep. 19, 2024.
(60) Provisional application No. 63/842,575, filed on Jul. 11, 2025.

(51) Int. Cl. A61K 31/415; A61P 19/00; A61P 21/00

(52) U.S. Cl. CPC A61K 31/415; A61P 19/00; A61P 21/00

(57) ABSTRACT

The present disclosure relates to an alternative intervention to improve skeletal muscle health in chronic kidney disease (CKD). CKD causes skeletal muscle complications that lead to increased risk of falls, hospitalizations and mortality. While exercise is a common treatment for these impairments in the general population, in patients with CKD exercise has shown only marginal improvements in aerobic capacity and inconsistent effects on muscle strength, walking distance, and timed-up-and-go tests indicating this ‘usual’ treatment is not enough in patients in CKD. The present disclosure relates to methods of using an aminoindazole to treat inflammation and metabolic derangements and to improve skeletal muscle health in CKD.

### RELATED APPLICATIONS

This application claims the benefit of U.S. Provisional Application No. 63/842,575, filed Jul. 11, 2025, and U.S. Provisional Application No. 63/696,443, filed Sep. 19, 2024, the entire disclosure of each of which is incorporated herein by reference.

### BACKGROUND

Chronic kidney disease (CKD) leads to progressive deterioration of skeletal muscle and bone leading to sarcopenia and CKD-bone mineral disease (CKD-MBD). Exercise studies have demonstrated variable success, which supports the need for pharmacological interventions. Muscle and bone problems plague patients with chronic kidney disease. Currently there are no drug therapies that beneficially impact muscle, and bone therapies are limited with side effects. Exercise is commonly employed for those with muscle and bone impairments, but in CKD, the evidence is inconsistent with small to moderate effects.

Chronic kidney disease (CKD) causes skeletal muscle complications that lead to increased risk of falls, hospitalizations and mortality. While exercise is a common treatment for these impairments in the general population, in patients with CKD exercise has shown only marginal improvements in aerobic capacity and inconsistent effects on muscle strength, walking distance, and timed-up-and-go tests. This indicates the ‘usual’ treatment of exercise is not enough in patients in CKD2, Preclinical studies also show similar inconsistencies across different exercise modes and intensities.

CKD progression can lead to muscle weakness alone (dynapenia) or combined with muscle atrophy (sarcopenia). Muscle strength and function are consistently reduced by 20-35% in clinical and preclinical subjects compared to healthy controls. The prevalence of sarcopenia in CKD populations varies from 4-63% due to variability in diagnostic definitions and criteria. Recent meta-analysis focused on muscle atrophy in 106 clinical and preclinical CKD studies found small-to-moderate effect sizes for muscle atrophy in clinical cohorts (0.48) and larger effect sizes in preclinical studies (0.95). The Cy/+ model was not included in the meta-analysis due to lower than three studies, but it demonstrated muscle atrophy similar to the clinical findings. Further, sex differences were identified in the meta-analysis with greater atrophy in male subjects and animals. These findings underscore that muscle dysfunction is prominent in CKD.

Additionally, metabolic defects and inflammation are common in CKD. CKD causes systemic inflammation and alterations of multiple metabolic pathways leading to multi-organ complication. CKD leads to a variety of metabolic derangements including 1) accumulation of toxins from decreased kidney clearance, 2) abnormal metabolism by the kidney impacting protein and lipid abnormalities, 3) increased oxidative stress and mitochondrial metabolism, 4) alterations in mineral metabolism and acid/base homeostasis and 5) changes in chromatin. These changes are progressively more severe and varied with progressive CKD and not corrected with dialysis. The data presented herein suggests mitochondrial, lipid oxidation, mineral (calcium, phosphorus) metabolism, and inflammation are altered in the muscle of preclinical models.

CKD leads to accumulation of uremic toxins driving systemic inflammation, decreased renal clearance and impaired cellular metabolism in the kidney and systemic tissues. Accumulated toxins disrupt enzyme function, acid base balance, and other metabolic processes. In skeletal muscle, CKD-induced metabolic impairments reduce the use of free fatty acids through β-oxidation, leading to fatty acid accumulation and conversion to triglycerides. Triglycerides rich in long-chain and saturated fatty acids impose greater mitochondrial demand through the required carnitine shuttle; this shuttle is impaired in CKD due to a carnitine deficiency. Researchers have attempted to correct the metabolic defect with carnitine supplementation, but supplementation showed no beneficial effects and, in some cases, worsened renal disease. These findings motivate the exploration of alternative interventions to improve metabolic defects that impact skeletal muscle health in CKD.

Inflammation is another contributing factor in CKD. CKD is characterized as a chronic, low-grade inflammatory state that leads to mitochondrial defects causing muscle weakness, decreased exercise tolerance, and worsening physical health. In CKD, the accumulation of uremic toxins, such as indoxyl sulfate and p-cresyl sulfate, stimulate inflammatory pathways that lead to the overproduction of pro-inflammatory cytokines. These cytokines are also prevalent in metabolic disorders including type II diabetes, metabolic syndrome, and obesity. The cyclical relationship between inflammation and metabolic dysregulation in CKD creates a feedback loop of high levels of oxidative stress driving the production of reactive oxygen species (ROS) (i.e., malondialdehyde, isoprostanes, 8-hydroxy-2-deoxyguanosine (8-OHdG)) that serve as a pro-inflammatory stimulus to increase gene expression of growth factors, inflammatory cytokines, and chemokines. The link between inflammation and metabolism in CKD support a pathway with promise to improve skeletal muscle health. The roles of chronic inflammation and mitochondrial dysfunction in CKD are not fully understood. Accordingly, therapeutics and approaches to investigate for CKD are needed.

### BRIEF DESCRIPTION OF THE DRAWINGS

FIGS. 1A-1I demonstrate impaired kidney function, abnormal mineral metabolism, and increased aorta calcification in CKD rats compared to normal rats (FIGS. 1A-1F). Locamidazole (LAMZ) treatment reduced parathyroid hormone (PTH) (FIG. 1A) and trended towards improving muscle strength (FIG. 1B) in CKD rats but had no significant impact on other measures in CKD rats (1C-1I).

FIG. 2 shows a forest plot of acylcarnitine levels in the soleus and extensor digitorum longus EDL. Acylcarnitines fold change was lower in CKD compared to normal littermates (NL) in the soleus (left) and variable in the EDL (right); NL and CKD comparison. Dot size indicates p-value, largest dots (p<0.005), medium dots (p<0.05) and small dots (p>0.05).

FIG. 3 shows a PET scan of impaired fatty acid metabolism. Biodistribution studies on excised EDL and soleus show statistically significant (p<0.05) decreases in ¹¹C-palmitate uptake.

FIG. 4 shows that LAMZ partially improved mitochondria morphology. TEM images of the EDL from NL, CKD, and CKD+LAMZ at 35 weeks of age. NL-normal mitochondria circular in shape with defined borders (circled); CKD-misshaped mitochondrial with an onion like appearance that is an indicator of mitophagy (circled). LAMZ-improved multiple mitochondria (circled), with some persistent derangements of misshaped and onion-like appearance (compare circled area versus squared area). The partial improvement of mitochondria supports a longer treatment duration.

FIGS. 5A-5D show LAMZ restored muscle strength and improved muscle fatigue and maximal running capacity. CKD impaired fatigue (FIGS. 5A, 5B), strength (FIG. 5C), porosity (FIG. 5D), and thickness that was improved via LAMZ. *=p<0.05, **=p<0.01.

FIGS. 6A-6D show that LAMZ lowers PTH without impacting disease progression. LAMZ did not impact BUN (FIG. 6A), FGF23 (FIG. 6B), or creatinine (FIG. 6C); LAMZ reduced PTH (FIG. 6D).

FIGS. 7A and 7B show LAMZ regulates inflammatory and oxidative pathways in the soleus. mRNAseq was performed with dot plots depicting both magnitude (size) and color (p-value) for CKD-induced suppression of oxidative pathways and activation of inflammatory pathways (FIG. 7A). LAMZ activated oxidative and suppressed inflammatory pathways (FIG. 7B).

FIG. 8 shows IHC staining for macrophage marker ED2 of the EDL. Representative images demonstrate LAMZ inhibited a CKD-induced increase in macrophage expression; as stained for the ED2 marker.

### DETAILED DESCRIPTION OF THE DISCLOSURE

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. Sec, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein can be derived using commercially-available software such as ACD/Name 2014 (ACD/Labs) or ChemBioDraw Ultra 13.0 (Perkin Elmer).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

As used herein, unless otherwise indicated, the term “treating” means reversing, alleviating, inhibiting the progress of (i.e., curative treatment), or preventing the disorder or condition to which such term applies, or one or more symptoms of such disorder or condition. The term “treatment”, as used herein, unless otherwise indicated, refers to the act of treating as “treating” as defined immediately above. “Preventative” treatment is meant to indicate a postponement of development of a disease, a symptom of a disease, or medical condition, suppressing symptoms that may appear, or reducing the risk of developing or recurrence of a disease or symptom. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. “Curative” treatment includes reducing the severity of or suppressing the worsening of an existing disease, symptom, or condition. Thus, treatment includes ameliorating or preventing the worsening of existing disease symptoms, delay or slowing of disease progression, amelioration or palliation of the disease state, and remission (whether partial or total), whether detectable or undetectable, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. “Treatment” can also mean prolonging survival as compared to expected survival if not receiving treatment.

As used herein, the term “therapeutically effective amount” refers to that amount of active compound or pharmaceutical agent that elicits the biological or medicinal response in a patient, which includes alleviation of the symptoms of the disease or disorder being treated. In one aspect, the therapeutically effective amount is that which may treat or alleviate the disease or symptoms. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors. An exemplary dose is in the range of about from about 0.1 mg to 1.5 g daily, about 0.1 mg to 1 g daily, or about 1 mg to 50 mg daily, or about 50 to 250 mg daily, or about 250 mg to 1 g daily. The total dosage may be given in single or divided dosage units (e.g., QD, QW, BID, TID, QID).

A “patient,” “subject,” “host animal,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

As used herein, the term “pharmaceutically acceptable salt” refers to those salts which counter ions which may be used in pharmaceuticals. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt. Such salts include:
(1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or
(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.

Pharmaceutically acceptable salts are well known to those skilled in the art, and any such pharmaceutically acceptable salt may be contemplated in connection with the embodiments described herein. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene 2sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof. For example, it will be appreciated that compounds depicted by a structural formula containing the symbol “ [symbol] ” include both stereoisomers for the carbon atom to which the symbol “ [symbol] ” is attached, specifically both the bonds “ [symbol] ” and “ [symbol] ” are encompassed by the meaning of “ [symbol] ”.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. For example, isotope-labeled compounds and salts can be used as medicaments. Further, substitution with heavier isotopes such as deuterium (i.e., ²H) may afford certain therapeutic advantages resulting from greater metabolic stability, for example increased in vivo half-life or reduced dosage requirements. For example, deuterium (²H)-labeled compounds and salts may be therapeutically useful with potential therapeutic advantages over the non-²H-labeled compounds. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

These definitions are meant to clarify, but not limit, the terms defined. If a particular term used herein is not specifically defined, such term should not be considered indefinite. Rather, terms are used within their accepted meanings.

### Illustrative Embodiments

Understanding the role of inflammation in CKD is important as inflammation directly impairs skeletal muscle. Inflammatory myopathies are characterized by inflammation that activates immune cells which directly damage myofibers leading to atrophy and weakness. Muscle atrophy was considered a secondary consequence of inflammation; however, it has been shown that skeletal muscle has direct relationships with the innate immune response through the NLRP3 inflammasome. Activation of NLRP3 is two-stage process of priming that activates the transcription of NLRP3, pro-caspase-1, pro-IL-1β, and pro-IL-18. Transcription activation then signals trigger pathogen-associated molecular patterns (PAMPs) and damage-associated molecular patterns (DAMPs) that activate the sensor protein NLRP3, and the adaptor protein ASC (also known as PYCARD). Some data that supports activation of the innate immune system is activation of NLRP3 pathway, genes regulating macrophages, and muscle-specific macrophage staining.

One drug, Locamidazole (LAMZ) is a compound for improving skeletal muscle. LAMZ is a small molecule drug that has shown promise in preclinical studies by improving muscle strength, and size. This drug belongs to a class of receptor tyrosine kinase (RTK) inhibitors that target multiple RTKs including vascular endothelial growth factor receptors (VEGFR), platelet-derived growth factor receptor (PDGFR), and colony-stimulating factor 1 receptor (CSF1R). Results described in the present application demonstrate that LAMZ improves muscle function and mitochondrial morphology by regulating inflammatory and metabolic signaling and reducing parathyroid hormone (PTH) levels. LAMZ could be promoting these effects by counteracting CKD-induced skeletal muscle deterioration through suppression of inflammation and activation of metabolic pathways. This application demonstrates that LAMZ effects are mediated by both inflammatory and metabolic changes, demonstrates the role of PTH, and determines the optimal timing of intervention to maximize benefits.

In addition, LAMZ shows metabolic potential. LAMZ has been suggested to activate calcium signaling in skeletal muscle in a similar manner as exercise by stimulating gene expression through calcineurin and Ca²⁺/calmodulin-dependent protein kinase II (CAMKII). This signaling cascade commonly increases mitochondrial content via myocyte enhancer factor 2 (MEF2) and subsequent activation of PGC-1α, a key regulator of muscle metabolism and fatty acid oxidation. In CKD, lipid metabolism dysregulation contributes to disease progression and has been ameliorated via PGC-1α upregulation in acute kidney injury and aldosterone-induced podocytes injury. Although PGC-1α is a potential downstream target of LAMZ, data in the present application demonstrate that LAMZ beneficially affects skeletal muscle and mitochondria despite no change in PGC-1α gene expression. These changes with no effect on PGC-1α indicate a need for identifying additional mechanisms that regulate muscle health in CKD.

LAMZ also shows anti-inflammatory potential that could serve to ameliorate CDK symptoms or comorbidities. LAMZ belongs to a class of receptor tyrosine kinase (RTK) inhibitors that includes Linifanib, an inhibitor of multiple RTKs including VEGRF, PDGFR and CSF1R that contribute to the recruitment and chemotaxis of monocytes, macrophages, neutrophils to inflammatory sites, and thus enhance the inflammatory response. CSF1R inhibitors have been used in preclinical studies to deplete macrophages and used as an anti-necroptosis agent for the inflammatory condition of sepsis that reduced expression of the cytokine IL-6. In the disclosed CKD rat model, LAMZ effectively disrupted the pro-inflammatory cascade, suppressed immune cell-related gene expression, and improved muscle function (e.g., muscle strength).

LAMZ effects may be attributed to reduced parathyroid hormone (PTH). In CKD, renal deterioration leads to phosphate retention and decreased vitamin D which in turn stimulates PTH production. Elevated PTH accelerates muscle proteolysis, impairs mitochondrial function, and is associated with inflammation markers (e.g., IL-6, CRP, TNF). PTH is associated with high-density lipoprotein ratios and is a predictor of metabolic syndrome. In a large study of 4,322 men and 4,626 women from the National Health and Nutrition Examination Survey, IL-6 and CRP were positively associated with serum concentrations of PTH. The improved metabolic effects that are demonstrated herein occur despite no change in PGC-1α, but a 50% reduction in PTH. In vitro and in vivo studies will identify if PTH is an underlying disease-related factor that is central to LAMZ's benefits.

Consequently, the limited efficacy of exercise highlights the need for alternative treatments to improve muscle health and function in CKD. Accordingly, the disclosure provides use of locamidazole (LAMZ) to overcome inflammation and metabolic derangements to improve skeletal muscle health in CKD.

In certain aspects, an aminoindazole (e.g., locamidazole (LAMZ) or linifanib) shows muscle and bone anabolic effects that mimic exercise. LAMZ is a small molecule drug that has been previously described to enhance myogenesis and osteoclastogenesis leading to improved bone and muscle mass. In certain embodiments, administration of an aminoindazole (e.g., locamidazole (LAMZ) or linifanib) in a preclinical model of CKD leads to improved muscle strength and fatigue mitigating the effects of the disease. In some embodiments, an aminoindazole (e.g., locamidazole (LAMZ) or linifanib) improves physical function and bone outcomes in a rat model of CKD.

In certain aspects, an aminoindazole (e.g., locamidazole (LAMZ) or linifanib) improves locomotor function in normal aging.

In certain aspects, a method of improving skeletal health and/or muscle strength in a patient having chronic kidney disease, comprises administering a therapeutically effective amount of an aminoindazole to the patient in need thereof.

In some embodiments, the aminoindazole is

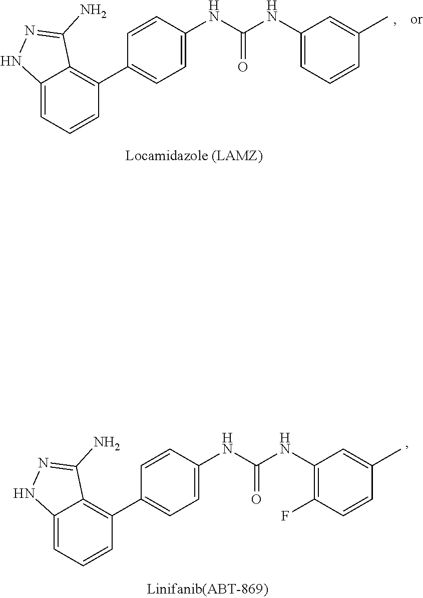 [SMILES: Cc1cccc(NC(=O)Nc2ccc(-c3cccc4[nH]nc(N)c34)cc2)c1 ; Cc1ccc(F)c(NC(=O)Nc2ccc(-c3cccc4[nH]nc(N)c34)cc2)c1]

or a pharmaceutically acceptable salt thereof, and preferably is locamidazole. In some preferred embodiments, the aminoindazole is LAMZ, or a pharmaceutically acceptable salt thereof. In some embodiments, the aminoindazole is linifanib, or a pharmaceutically acceptable salt thereof.

In some aspects, a method of improving skeletal health and/or muscle function in a patient experiencing muscle wasting comprises:
administering a therapeutically effective amount of an aminoindazole (e.g., locamidazole (LAMZ) or linifanib), or a pharmaceutically acceptable salt thereof, to the patient in need thereof.

In some aspects, a method of improving skeletal health and/or muscle function in a patient having chronic kidney disease, comprises:
administering a therapeutically effective amount of an aminoindazole (e.g., locamidazole (LAMZ) or linifanib), or a pharmaceutically acceptable salt thereof, to the patient in need thereof.

In some embodiments, the method improves the handgrip strength, the sit-to-stand test, the 6-minute walk test, the timed up and go test, the gait speed, the isometric knee extensor strength, the short physical performance battery scores, or a combination thereof.

In some embodiments, the method improves muscle function by muscle strength and/or muscle fatigue. In some embodiments, the method improves skeletal health by increasing bone cortical thickness and/or porosity. In some embodiments, the patient has an amyotrophic disease.

In some embodiments, the method downregulates inflammatory pathways. In some embodiments, the method reduces PTH levels. In some embodiments, the method normalizes macrophage expression. In some embodiments, the method reduces macrophage infiltration in skeletal muscle. In some embodiments, the method upregulates a gene in a metabolic pathway.

In some embodiments, the metabolic pathway is selected from the group consisting of fatty acid oxidation, ATP synthesis, and the electron transport chain. In some embodiments, the method improves mitochondrial morphology. In some embodiments, the mitochondrial morphology comprises reduced mitochondrial swelling and/or reduced disorganization of mitochondrial cristae. In some embodiments, the mitochondria impairment is decreased when compared to a patient not receiving the aminoindazole (e.g., locamidazole (LAMZ) or linifanib). In some embodiments, the aminoindazole improves fatty acid uptake and esterification.

In some embodiments, the aminoindazole is administered in combination with an additional therapeutic. In some embodiments, the aminoindazole and the additional therapeutic (e.g., an anti-inflammatory agent) are co-formulated. In some embodiments, the aminoindazole and the additional therapeutic (e.g., an anti-inflammatory agent) are administered at the same time. In some embodiments, the aminoindazole and the additional therapeutic (e.g., an anti-inflammatory agent) are individually formulated and administered at the same time. In some embodiments, the aminoindazole and the additional therapeutic (e.g., an anti-inflammatory agent), and administered in sequence. In some embodiments, the sequential administration of the aminoindazole and the additional therapeutic (e.g., an anti-inflammatory agent) can be accomplished with the aminoindazole administered first, and the additional therapeutic (e.g., an anti-inflammatory agent) administered second. In some embodiments, the sequential administration of the aminoindazole the additional therapeutic (e.g., an anti-inflammatory agent) can be accomplished with the additional therapeutic (e.g., an anti-inflammatory agent) administered first, and the aminoindazole administered second.

In some embodiments, the additional therapeutic is a GLP-1 inhibitor. In another embodiment, the additional therapeutic is an anti-inflammatory drug. In another embodiment, the additional therapeutic is a Non-Steroidal AntiInflammatory (NSAID) or steroid. In some embodiments, the additional therapeutic is an NSAID.

In another aspect, a method of improving skeletal health and/or muscle function in a patient experiencing muscle wasting comprises inhibiting CSF1R, VEGF, KDR, PDGF, or a combination thereof. For example, in some embodiments, the step of inhibiting comprises administering a therapeutic that inhibits CSF1R, VEGF, KDR, PDGF, a combination thereof, or a pathway involving CSF1R, VEGF, KDR, PDGF, or a combination thereof. In some embodiments, the step of inhibiting comprises administering a therapeutic, for example an aminoindazole (e.g., LAMZ), KP-2326, PLX5622, or any combination thereof.

### Additional Embodiments

1. A method of improving skeletal health and/or muscle function in a patient experiencing muscle wasting, the method comprising:
administering a therapeutically effective amount of an aminoindazole, or a pharmaceutically acceptable salt thereof, to the patient in need thereof.
   2. A method of improving skeletal health and/or muscle strength in a patient having chronic kidney disease, the method comprising:
administering a therapeutically effective amount of an aminoindazole, or a pharmaceutically acceptable salt thereof, to the patient in need thereof.
   3. The method of embodiment 1 or 2, wherein the aminoindazole is locamidazole, or a pharmaceutically acceptable salt thereof, or linifanib, or a pharmaceutically acceptable salt thereof.
   4. The method of embodiment 1, 2, or 3, wherein the method improves the handgrip strength, the sit-to-stand test, the 6-minute walk test, the timed up and go test, the gait speed, the isometric knee extensor strength, the short physical performance battery scores, or a combination thereof.
   5. The method of embodiment 1, 2, or 3, wherein the improvement of muscle function is muscle strength and/or muscle fatigue.
   6. The method of embodiment 1, 2, or 3, wherein the method improves skeletal health by increasing bone cortical thickness and/or porosity.
   7. The method of any one of embodiments 1-6, wherein the method downregulates inflammatory pathways.
   8. The method of embodiment 7, wherein the method reduces PTH levels.
   9. The method of any one of embodiments 1-7, wherein the method normalizes macrophage expression.
   10. The method of any one of embodiments 1-7, wherein the method reduces macrophage infiltration in skeletal muscle.
   11. The method of any one of the preceding embodiments, wherein the method upregulates a gene in a metabolic pathways, improves fatty acid uptake and esterification, or both.
   12. The method of embodiment 11, wherein the metabolic pathway is selected from the group consisting of fatty acid oxidation, ATP synthesis, and the electron transport chain.
   13. The method of any one of the preceding embodiments wherein the method improves mitochondrial morphology.
   14. The method of embodiment 13, wherein the improved mitochondrial morphology comprises reduced mitochondrial swelling and/or reduced disorganization of mitochondrial cristae.
   15. The method of any one of the preceding embodiments, wherein mitochondria impairment is decreased when compared to a patient not receiving the aminoindazole.
   16. The method of any one of the preceding embodiments, wherein the aminoindazole is locamidazole, or a pharmaceutically acceptable salt thereof.
   17. The method of any one of the preceding embodiments, wherein the aminoindazole is administered in combination with an additional therapeutic.
   18. The method of embodiment 17, wherein the additional therapeutic is a GLP-1 inhibitor.
   19. The method of embodiment 17, wherein the additional therapeutic is an anti-inflammatory drug.
   20. The method of embodiment 17 or 19, wherein the additional therapeutic is a NSAID or steroid.
   21. The method of any one of embodiments 17, 19, or 20 wherein the additional therapeutic is a NSAID.
   22. The method of any one of the preceding embodiments, wherein the patient has an amyotrophic disease.

### EXAMPLES

### Example 1

### Locamidacole (LAMZ) Restores Muscle and Bone Outcomes in a Rat Model of Chronic Kidney Disease (CKD).

A naturally occurring and progressive rat model of CKD (Cy/+; n=10-12 gr) with normal littermates (NL), a CKD model (Cy/+), CKD treatment (CDK-LAMZ). LAMZ was injected subcutaneously (s.q.), 2× per day at 0.625 mg/kg for 5 weeks, beginning at 27 weeks (CKD stage 3). At 33 weeks (end-stage renal disease), rats were terminated for specimen collection.

The disease effects for CKD compared to NL was found via elevated blood urea nitrogen (BUN; p<0.0001) and creatinine (p<0.0001), muscle weakness (p<0.05), muscle fatigue (p<0.01), lower running distance (p<0.05), increased cortical bone porosity (p<0.001), reduced cortical bone area (p<0.01), and cortical thickness (p<0.001). The animals treated with LAMZ indicated that LAMZ did not impact CKD progression, but LAMZ did improve muscle function with significantly improved maximally stimulated muscle strength (p<0.05) with treating improvements for electrically stimulated muscle fatigue (p=0.08). Additionally, LAMZ improved bone health with reduced cortical porosity (p<0.01) and increased cortical thickness (p<0.01). Maximal running distance demonstrated an intermediate level of adaptation (i.e., not statistically different from NL or CKD)

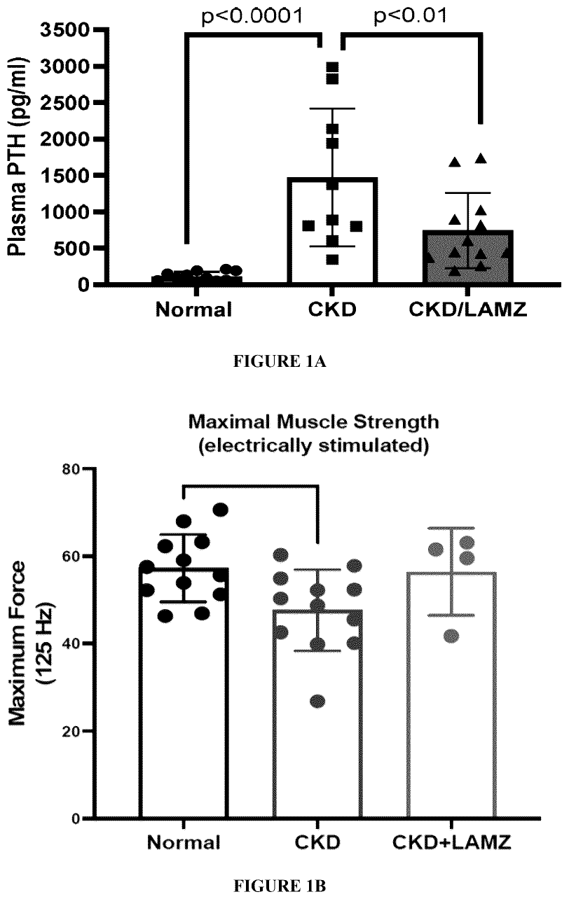

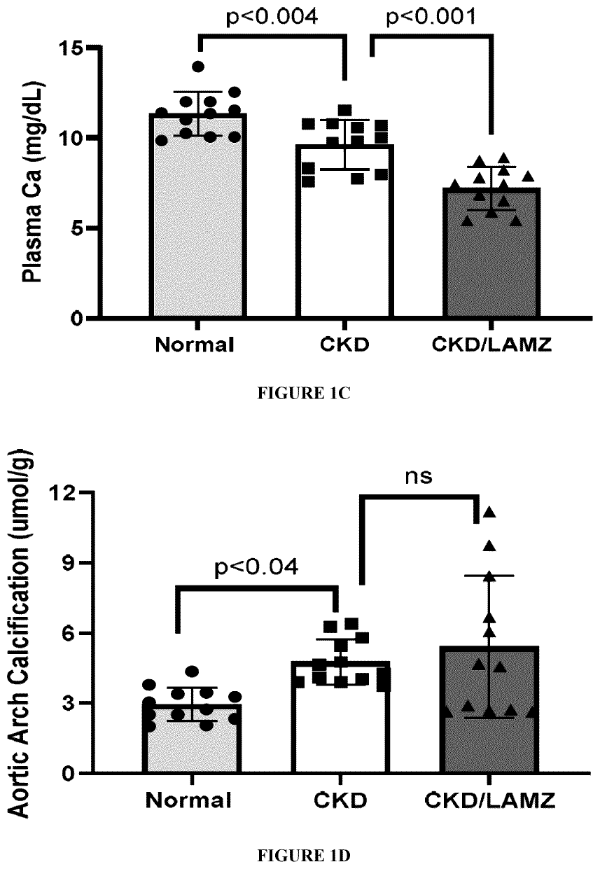

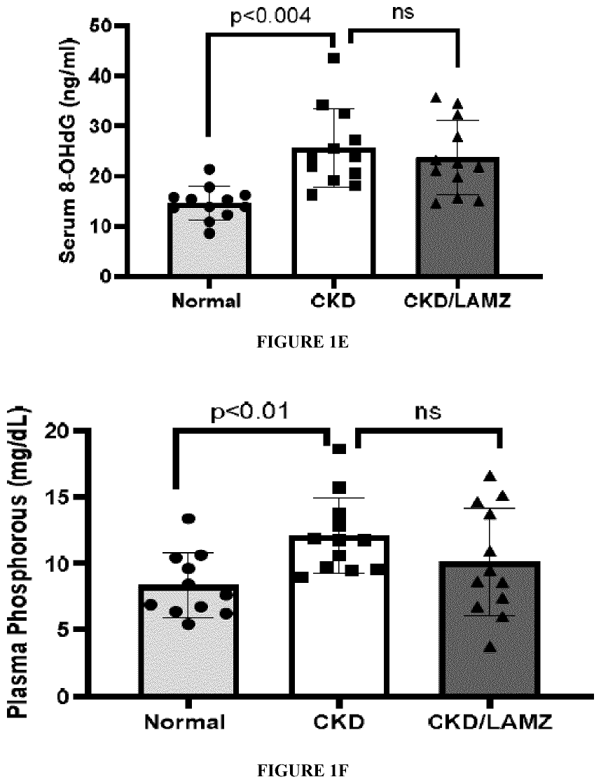

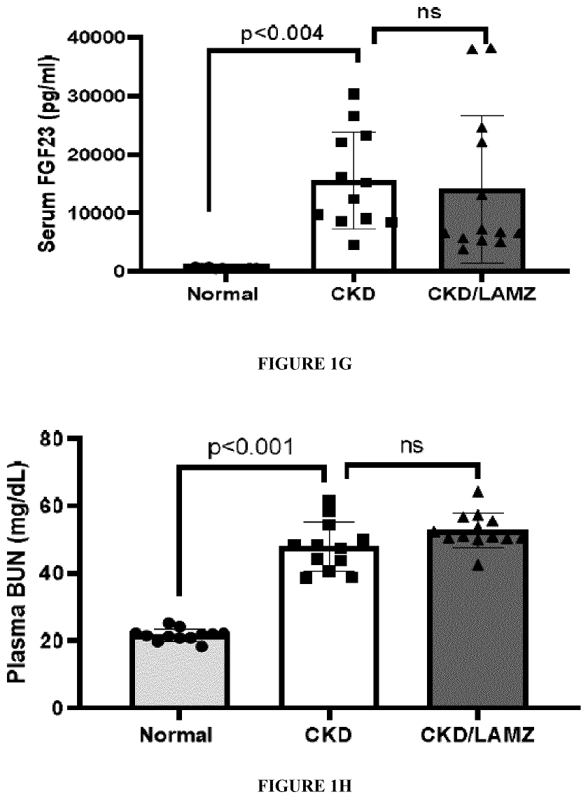

Results from the study demonstrate impaired kidney function, abnormal mineral metabolism and aorta calcification in CKD rats compared to normal rats (FIGS. 1A-1I). LAMZ treatment reduced PTH (FIG. 1A) and trended towards improving muscle strength (FIG. 1B) in CKD rats but had no significant impact on other measure in CDK rats (FIGS. 1C-1F). From the data, the study indicates that LAMZ administered for 5 weeks significantly improved both muscle strength and bone microarchitecture.

### Example 2

### CKD Impairs Skeletal Muscle Metabolism.

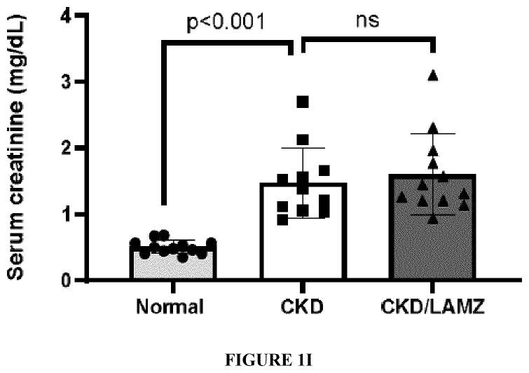

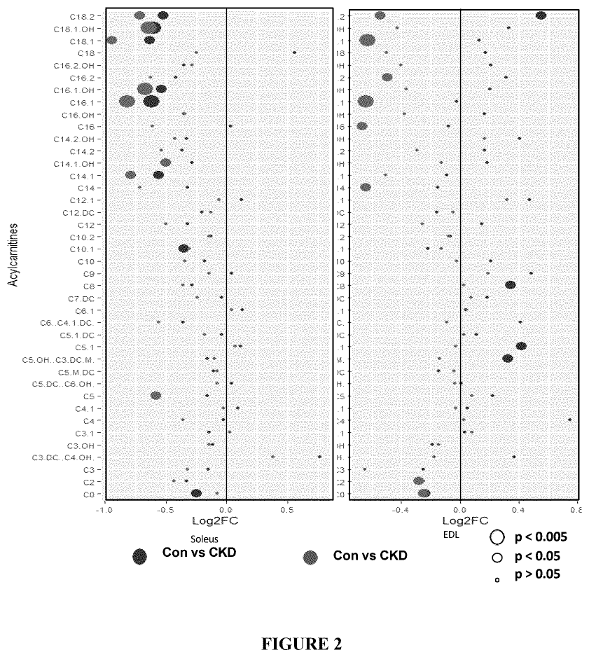

Targeted metabolomic analysis from muscle demonstrated the soleus had 33 significantly different metabolites between normal littermate (NL) and chronic kidney disease (CKD). A third of the alterations were reduced acylcarnitines, a trend that was mirrored in the plasma (FIG. 2). The extensor digitorum longus (EDL) demonstrated variable results; however, carnitine (CO) was significantly reduced in both muscles and plasma (not shown).

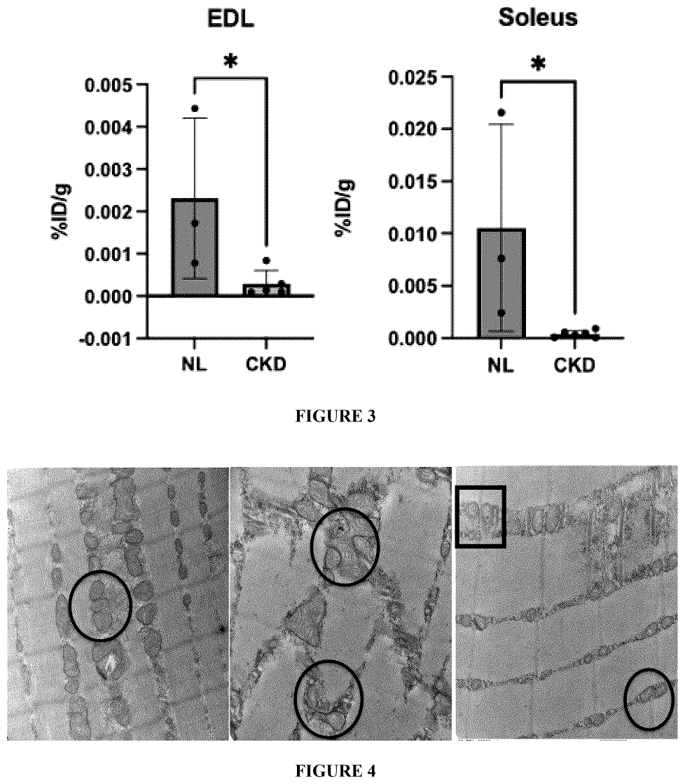

To further interrogate altered fatty acid metabolism, in vivo dynamic PET/CT imaging with ¹¹C-Palmitate. Kinetic analyses revealed lower fatty acid uptake and esterification rates in both limb and paraspinal muscles of CKD rats compared to normal littermates. Confirmatory biodistribution studies in excised muscle tissues corroborated these findings, showing reduced ¹¹C-palmitate uptake in both EDL and soleus muscles normalized per gram of tissue (FIG. 3). To address carnitine deficiency, a 10-week course of carnitine injections were administered, which unexpectedly worsened kidney function and serum biomarkers of mineral metabolism and oxidative stress without improving musculoskeletal function. The data provides direct and indirect evidence of impaired metabolism that underscores the need for interventions addressing multiple pathways in CKD.

### LAMZ Lowered PTH and Improved Skeletal Muscle Mitochondria and Dynapenia in CKD Rats.

In a five-week study, doses of LAMZ ranged from 0.0625, 0.1, 0.625, 1.25, 2.5, and 5.0 mg/kg 2× daily; n=2-5/group. Higher doses (2.5 and 1.25 mg/kg 2× daily) led to gastrointestinal side effects and early mortality; lower doses (0.0625 mg/kg and 0.1 mg/kg) lacked functional efficacy.

The 0.625 mg/kg dose was well-tolerated and normalized muscle strength, while improving mitochondrial morphology and muscle fatigue. To determine if renal disease does not impact LAMZ metabolism and clearance, a pharmacokinetic analysis was performed using 0.625 mg/kg LAMZ injected at 30 weeks of age (30% of normal renal function for CKD) using standard noncompartmental analyses (NCA) methods.

There were no differences between CKD and normal littermates for half-life, volume of distribution, and clearance. These data identified a therapeutic dose and indicated that LAMZ was not renally cleared, and therefore not impacted by declining renal function.

Mitochondrial Morphological Defects are Partially Improved with LAMZ.

Mitochondrial morphology was assessed in the EDL (FIG. 4) and soleus (not shown) from normal littermates, CKD, and CKD+LAMZ (0.625 mg/kg for five weeks).

An independent and blinded assessment was performed which reported similar findings in both EDL and Soleus. The normal EDL demonstrated ordinary mitochondrial ultrastructure with typical size, contained and well-defined membrane, and well-delineated cristae arrangements. In CKD EDL, the mitochondria appeared swollen with disorganized and fragmented cristae, showing extensive mitochondrial lesions, loss of inner and outer membranes, and a reduction in mitochondria density. Abnormal mitochondrial structures included the destruction of cristae with expanded matrix space, concentric ‘onion shaped’, and densely compacted cristae. LAMZ-treated CKD rats demonstrated significantly reduced mitochondrial swelling and disorganization of cristae as compared to CKD mitochondria. (FIG. 4) It is noted the magnification for all images is 230000×; CKD and CKD+LAMZ images were digitally zoomed to allow improved observation of structural changes more closely, making these images appear more ‘zoomed in.’ This approach allows for highlighting the specific details relevant to each condition.

### LAMZ Improved Dynapenia in a Rat Model of CKD.

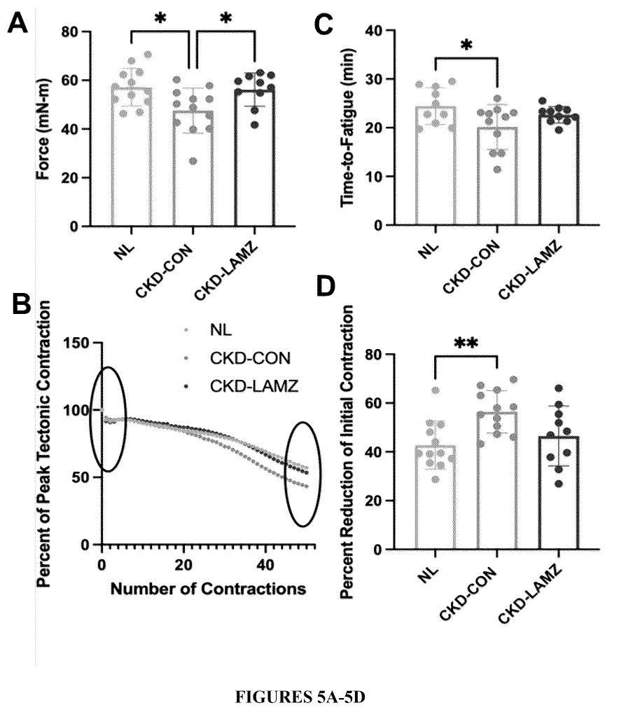

Five weeks of 0.625 mg/kg LAMZ normalized muscle strength and trended towards improved fatigue (n=10-12/group). Maximal electrically stimulated force of the dorsiflexor muscles assessed at 125 Hz, demonstrated a disease-induced reduction that was normalized with LAMZ treatment (FIG. 5A). Muscle fatigue was assessed by fifty repeated electrical stimulations of the dorsiflexor muscles at 60 Hz and recording force decline over time. A linear mixed model analysis demonstrated CKD rats fatigued faster than normal littermates with a slope of −1.192 versus −0.783, respectively. LAMZ (slope=−0.952) rats fatigued at a slightly faster rate than normal littermates, but less than CKD alone (FIG. 5B). Percent change in force production at the 50th contraction demonstrated greater fatigue in CKD compared to normal (p<0.05), while LAMZ compared to CKD was trending (p=0.07) (FIG. 5D). Maximal running capacity, depicted as time-to-fatigue, was a graded protocol that demonstrated a CKD-induced reduction compared to NL (p<0.05), but LAMZ was not able to restore this aspect of physical function (p=0.28) (FIG. 5C). Muscle endurance and fatigue resistance were partially improved.

LAMZ Lowered PTH without Impacting CKD Progression.

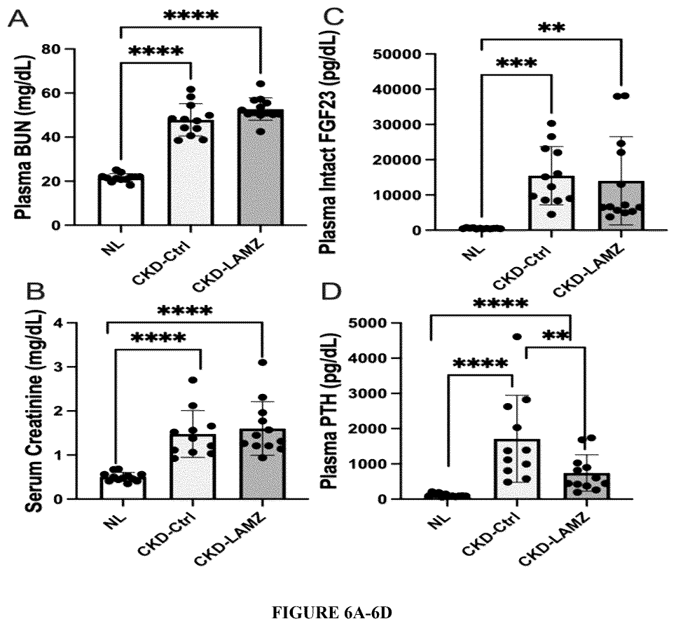

While LAMZ did not alter CKD progression markers (BUN, creatinine), it reduced PTH levels by 50% (FIGS. 6A-6D). Elevated PTH is associated with increased inflammation with increased cytokines and expression of inflammatory markers. The data demonstrates PTH reduction coincided with suppression of inflammation and improved mitochondria and skeletal muscle.

Metabolic and Inflammatory Regulatory Genes and Pathways were Both Improved with LAMZ.

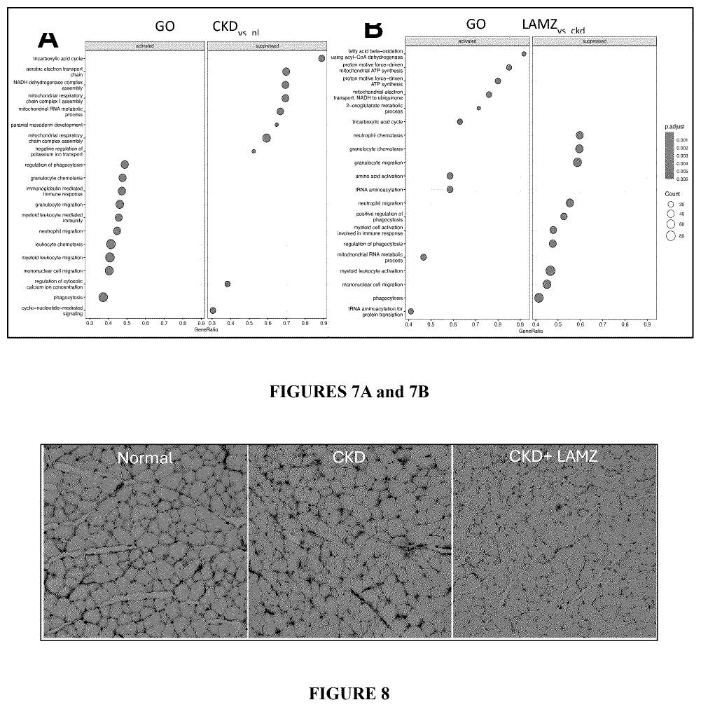

Metabolic pathways were identified through mRNAseq (n=6/gr) on the EDL and soleus from 35-week-old normal littermates, CKD, and CKD+LAMZ. The total number of genes identified with FDR p-value and log fold-change are depicted in Table 1. Gene Ontology (GO) pathway analysis demonstrated consistency between both the EDL (not shown) and soleus (FIG. 7A-B). Analyses revealed downregulation of oxidative pathways and upregulation of inflammatory pathways in CKD muscle. LAMZ treatment reversed this, downregulating inflammatory genes (NLRP3 [−1.92 log FC, p<0.05], and PYCARD [−1.18 log FC, p<0.05]) and upregulating pathways related to fatty acid oxidation and ATP synthesis, underscoring LAMZ's dual impact on inflammation and metabolism. Overall, LAMZ downregulated the inflammatory pathways and upregulated multiple genes related to metabolic pathways including fatty acid oxidation, ATP synthesis, and the electron transport chain, which supports the role of LAMZ in mediating metabolic and inflammatory pathways in CKD.

TABLE 1
Summary of mRNA Gene Ontology pathway analysis.
mRNAseq Gene Summary

| | | Total | | (−) LOG | (+) LOG |
|---|---|---|---|---|---|
| | Muscle | Genes | FDR <0.05 | FC | FC |
| CKD v. NL | EDL | 10521 | 549 | 33 | 79 |
| Lamz v. CKD | EDL | 10521 | 1781 | 351 | 12 |
| CKD v. NL | Soleus | 10931 | 771 | 27 | 369 |
| Lamz v. CKD | Soleus | 10931 | 801 | 575 | 5 |

### LAMZ Reduces Macrophage Infiltration in CKD Muscle.

Significant inflammation has been demonstrated in CKD compared to healthy controls in rat and mouse CKD models analogous to the Cy/+ and in adenine diet-induced CKD. Data herein shows plasma from 35-week-old Cy/+ rats (n=8) had significantly increased expression in multiple inflammatory cytokines including vascular endothelial growth factor (VEGF; p<0.0001), fractalkine (CX3CL1; p<0.01), interleukin-18 (IL-18; p<0.05), and monocyte chemoattractant protein-1, (MCP-1, CCL2; p<0.05 all of which can promote macrophage infiltration in muscle. To substantiate CKD-induced macrophage activation in the skeletal muscle, immunohistochemistry (IHC) staining for ED2 monoclonal antibody was performed (MCA342). Increased macrophage expression in CKD was found, which was normalized with LAMZ treatment (FIG. 8). Macrophages were specifically targeted as T-cell marker staining of CD3 and CD4 demonstrated no expression differences between groups. These data support the presence of inflammation in the muscle in CKD compared to normal animals. Furthermore, LAMZ reduced macrophages in the skeletal muscle.

Exercise interventions in CKD fail to consistently improve muscle health, underscoring the need for new therapeutic options. The data herein demonstrates that CKD activates pro-inflammatory signaling with increased plasma cytokines, suppression of oxidative pathways, mitochondrial derangements, and impaired skeletal muscle. LAMZ daily injections for five weeks normalized skeletal muscle strength, improved mitochondria morphology, and trended towards improving muscle fatigue. LAMZ did not impact kidney disease progression, but reduced PTH by 50%. PTH has a potential role in inflammation, and metabolic and muscle dysfunction. Gene profiling and IHC of the skeletal muscle supported the CKD-induced activation of macrophages that was also suppressed with LAMZ treatment.

### Example 3

### Determination of the Effects of LAMZ Treatment Duration on Skeletal Muscle Function, Morphology, and Metabolism in Two Different Models of CKD.

Data presented in Example 1 from the Cy/+ model indicated LAMZ treatment normalized muscle strength (p<0.05), with promising trends in muscle fatigue (p=0.07). In contrast to the effects in healthy and hindlimb unloaded mice, in the Cy/+ rat CKD model, PGC-1α did not show consistent impairment indicating pathways beyond PGC-1α that yield beneficial effects. Therefore, mRNAseq was employed. The mRNAseq analysis showed LAMZ-induced normalization of oxidative and mitochondrial-related pathways (FIGS. 7A and 7B) and improved mitochondrial morphology (FIG. 4). These improvements may be mediated by upregulation of key mitochondrial genes, including UGCC3 (complex III), Cox19 (complex IV), Oma1 (biogenesis), and Oxa1I (assembly), as identified in the mRNAseq data. While late LAMZ treatment improved muscle morphology, molecular signaling, and strength, full normalization was not achieved. This can be attributed to not starting analysis early enough in the course of CKD, or an insufficient treatment duration.

The studies in this example used three CKD models, 1) 1) Cy/+ male, 2) adenine-diet male, and 3) adenine-diet female. To control for potential background differences, healthy littermates will be used for the models 2 and 3 by administering the adenine to induce CKD. Within each of these models Normal, CKD, CKD+LAMZ early (beginning at 25 weeks of age), and CKD+LAMZ late (beginning at 30 weeks of age) will be compared. As previously noted, a therapeutic dose of 0.625 mg/kg was identified that will be administered daily via subcutaneous (s.q.) injections. All rats are bred in an in-house colony. At 24 weeks, a draw baseline blood draw will be performed, and the feed will change to begin a casein-based diet (TD.04539; 18% casein protein, 0.7% phosphate, 0.7% calcium) or casein-diet with the addition of 0.25% adenine (Envigo Teklad Diets, Madison, WI, USA). LAMZ will be injected subcutaneously two times per day for 10 and 5 weeks at 0.625 mg/kg (n=14/gr). At 33 weeks of age animals will undergo a PET scan to measure fatty acid metabolism, and at 34 weeks of age will undergo muscle strength and fatigue, and maximal running distance separated by 2-4 days. At 35 weeks rats will be terminated and tissue collection of both slow (soleus) and fast (EDL) fiber will be performed.

The primary outcomes include electrically stimulated maximal muscle strength, mitochondrial morphology, number and size (via electron microscopy), fatty acid PET imaging (¹¹C-palmitate or ¹⁸F-FTO-PET). Secondary outcomes include muscle fatigue, serum biochemistry, maximal running distance, metabolic signature via targeted metabolomics, muscle cross-sectional area, PCR and western blot for regulators of muscle atrophy (i.e., MyoD, myogenin, and Pax7). The only repeated outcome measures are muscle strength/fatigue/run at 24, 29 and 34 weeks of age. PET will be assessed at 34 weeks of age, with all of remaining assays will be performed post-mortem.

It will be expected earlier treatment of LAMZ will enable beneficial metabolic adaptations to occur prior to advanced renal progression, thus preventing mitochondrial derangement and metabolic impairments and therefore improving muscle. Similarly, it will be anticipated that the LAMZ-activated oxidative pathways demonstrated in the mRNAseq data will translate to improved fatty acid uptake and esterification as shown in PET imaging. If fatty acid uptake is unaffected by LAMZ, it could be due to a metabolic shift towards glycolysis or impairments in fatty synthesis and uptake. To account for metabolic shifts and flexibility, glucose metabolism will be assessed as well via ¹⁸F-FDG-PET (fluorodeoxyglucose-PET), as well as to explore signaling pathways for fatty acid synthesis. A metabolic signature or profile indicative of derangements identified in PET via metabolomics will be developed. It will be expected that both Cy/+ and adenine models will have reduced fatty acid utilization; however, it is possible that model differences could exist based upon published data of increased lipid-related metabolites in the adenine model. In contrast, the primary reduced lipid-related metabolites in the Cy/+ model have previously been identified. Further exploration into regulators of fatty acid pathways of synthesis, uptake, and esterification maybe be warranted and analyzed by PCR gene and western protein expression methods. No sex-differences within the adenine model are anticipated as CKD effects likely predominate over any sex differences. However, this needs to be tested as sex differences were found in a study of healthy Wistar rats where carnitine and multiple long-chain acylcarnitines were lower in male hearts compared to female hearts. However, in Sprague Dawley rats the converse occurred for a similar panel of acylcarnitines which were lower in the female rats. It is anticipated that mitochondrial changes occur independent of PGC-1α with changes in the mitochondrial dynamics of fission/fusion and mitophagy/apoptosis. Mitochondria morphology was partially restored following five week treatment with LAMZ, so it will be expected that 10 weeks of treatment with LAMZ will normalize morphology. If normalization occurs, the downstream citrate synthase activity, a marker for mitochondrial function, mitochondrial biogenesis, and regulators of mitochondrial oxidative phosphorylation (i.e., complex I-V, NADH Dehydrogenase, Uqccs, Cox 16/19, ATPsl, Tfam, Oma1, Oxa1I will be assessed.

### Elucidation of the Individual and Combination Effects of LAMZ-Induced Reduction of Inflammation and PTH on Restoring Skeletal Muscle Function in Two Mechanistically Distinct Models of CKD.

CKD is a state of chronic inflammation that coincides with an exponential increase in inflammation and PTH. The relationship between inflammation and PTH has been established by an association between disease progression/severity and PTH levels, as well as the reduction of cytokine expression in response to PTH suppressing drugs (i.e., calcimimetics). Proinflammatory cytokines are key mediators in the systemic inflammation that accelerates kidney damage and impacts multiple organ systems, including skeletal muscle. The result is chronic inflammation and mitochondrial dysfunction, but it remains uncertain as to whether the inflammation and metabolism are separate or linked phenomena, and what role PTH serves in this relationship. It has been demonstrated that LAMZ reduced CKD-induced macrophage expression which coincided with a reduction in PTH.

PTH has been shown to impact immune cells, where chronic exposure is accompanied by increases in both macrophages and T cells. Data presented from mRNAseq analysis, cytokine expression, and IHC (as shown in Example 2) supports the role of macrophages. Moreover, the data did not confirm T cell involvement. The role macrophages in the regulation of skeletal muscle size, strength and function by specifically depleting macrophages will be identified via PLX5622. PLX5622 is a selective small-molecule inhibitor of colony-stimulating factor 1 receptor (CSFIR), a crucial receptor for the survival, proliferation, and differentiation of macrophages. By inhibiting CSF1R signaling, PLX5622 effectively depletes macrophages in various tissues, including skeletal muscle. Macrophage depletion is expected to reduce inflammation (as will be assessed via cytokine panel) similar to LAMZ, without directly impacting PTH levels. This design will provide insight into how macrophage-driven processes influence muscle pathology and recovery in conditions characterized by chronic inflammation.

Additionally, the role of PTH in skeletal muscle dysfunction has been primarily demonstrated in those with secondary hyperparathyroidism with evidence of skeletal muscle weakness, dysfunction, and myopathies. Data presented herein demonstrated LAMZ reduced PTH which improved muscle health. PTH will be directly targeted via KP-2326. KP-2326 is a preclinical calcimimetic analogous to the clinically used etelcalcetide. KP-2326 directly activates the calcium sensing receptor to inhibit PTH release, regardless of the calcium level. By selectively reducing PTH activity, KP-2326 can be used to study the effects of diminished PTH levels in skeletal muscle. Reduced PTH signaling can impact muscle health by altering calcium regulation, muscle contraction, and metabolic processes. KP-2326 will be used to explore the role of PTH levels independent of inflammatory responses in CKD. Together evaluation of the effects of PLX5622 (inflammation) and KP-2326 (PTH) will establish if combined inhibition of these two pathways are needed to restore muscle health in CKD.

Therefore, it will be addressed if LAMZ could improve skeletal muscle function and metabolism through reduction of both inflammation and PTH, with additive effects. Additionally, LAMZ could have greater beneficial effects upon muscle function and metabolism as compared to singularly depleting macrophages or reducing PTH alone.

The complexity of inflammation and the underlying pathology of CKD limits facile identification of the role of PTH, and thus necessitates the use of in vitro experiments. In vitro experiments will be used to first identify the molecular targets of LAMZ as well as investigate the cellular effects of PTH. PTH is well known to differentially regulate bone health. Intermittent exposure of PTH has anabolic effects and chronic catabolic effects with increased bone resorption. Given bone and muscle are inextricably linked, the likelihood that PTH will have a similar effect in muscle would be expected. Previous work showed that acute, intermittent exposure of full-length PTH (1-84) on human satellite cells increased cAMP, myogenic differentiation gene expression and myosin-heavy chain protein expression. Interestingly, other research demonstrated that treatment with the PTH 1-34 fragment accelerated myogenesis and the production of myotubes in C2C12 mouse myoblast and ZHTc6-MyoD cells. Because the number of in vitro studies is limited and showed effects not aligned with bone studies and clinical studies of secondary hyperparathyroidism, especially given continuous exposure had beneficial effects in vitro the role of PTH in muscle is unclear. Consequently, the intermittent and continuous effect of PTH (both full-length (84) and fragmented (34) on differentiated muscle cells will be investigated.

### In Vivo Experimental Design.

To provide animals for the three models of Cy/+ male and adenine-diet male and female CKD rats, animals will be bred, weaned and placed on diet as described in Example 2. To determine the impact of inflammatory and PTH modulation has upon muscle function, the same groups within each of the three models will be studied. The five groups within each model will include 10 weeks of treatment for the following (n=14/gr): 1) NL, 2) CKD), 3) CKD+LAMZ, 4) CKD+KP-2326, and 5) CKD+PLX5622. The impact of each intervention on serum biochemistries including PTH, calcium, blood (BUN) nitrogen and creatinine will be assessed. Muscle function will be assessed as described in Example 2. Mitochondrial morphology will be assessed to determine the impact of manipulating the immune system versus interventions that can modulate PTH. A multi-cytokine panel will be utilized to assess the impact each intervention has on the inflammatory response. Inflammation will be further assessed via an 8-OHdG assay that indicates changes in oxidative stress. The cytokines will be appreciated in the context of proportional differences in immune cell populations in the whole blood, soleus and EDL by flow cytometry. Confirmation, using the same metabolic (including mitochondrial) and functional outputs described in Example 2.

### In Vitro Experimental Design.

In vitro experiments will be conducted to identify the effects of LAMZ, elucidate its pharmacological impact on tyrosine kinase pathways, and examine how uremic serum may modulate metabolic and inflammatory adaptations in skeletal muscle cells with and without LAMZ. To determine the specific tyrosine kinase pathways involved in LAMZ's effects, primary L6 (rodent) and human skeletal muscle cells will be used. Cells will be treated with control media or graded concentrations of LAMZ for 6, 24, and 48 hours. Targeted screening of tyrosine kinase pathways will be conducted using appropriate assays. If multiple pathways are implicated, selective inhibition using pharmacologic inhibitors and/or siRNA will be employed to assess the contribution of specific tyrosine kinase pathways. Calcium signaling will be measured using Fluo-4. To investigate the role of PTH, cells will be treated with control media or media containing 10% uremic serum with and without graded LAMZ concentrations. This experimental setup will allow the evaluation of myoblast differentiation, myotube size quantification, as well as PCR and western blot analyses for muscle differentiation markers (MyoD, myogenin, and Pax7). Inflammatory and metabolic adaptations will be assessed by measuring cytokine levels using a multiplex cytokine assay, conducting fatty acid uptake assays with tritium-labeled fatty acids, and performing western blot analysis for mitochondrial markers (PGC1α, TFAM). To confirm LAMZ's tyrosine kinase selectivity, targeted screening will be followed by siRNA knockdown of LAMZ targets to determine whether their absence affects myotube size. These experiments will provide a comprehensive analysis of LAMZ's role in modulating structural and metabolic adaptations in skeletal muscle cells under CKD conditions. By identifying cytokine profiles and specific LAMZ targets that influence these adaptations, these experiments will offer mechanistic insights into inflammation regulation and support translational progress for human therapeutic interventions.

This study includes serum biochemistries including PTH, blood urea nitrogen (BUN), creatinine, fibroblast growth factor 23 (FGF-23), and calcium levels. Electrically stimulated maximal muscle strength will be assessed, along with mitochondrial morphology, number, and size using electron microscopy, and a cytokine panel analysis will be performed to evaluate systemic inflammation. Secondary outcomes will involve fatty acid PET imaging (¹¹C-palmitate or ¹⁸F-FTO) to assess metabolic activity, flow cytometry to evaluate immune cell populations, and assessments of muscle fatigue and maximal running distance to determine functional endurance. Additionally, metabolic signature profiling using targeted metabolomics will be conducted, and muscle cross-sectional area will be measured. PCR and western blot analyses will be used to evaluate regulators of inflammation, including secreted phosphoprotein 1 (SPP1), chemokine (C-C motif) ligand 2, 7, 9 (CCL2, CCL7, CCL9), and arginase 1. Muscle strength, fatigue, and running endurance will be the only repeated outcome measures, assessed at 24, 29, and 34 weeks of age. PET imaging will be performed at 34 weeks of age, and all other assays will be conducted post-mortem. In vitro experiments will further assess the effect of LAMZ and PTH on skeletal muscle cells, focusing on myoblast differentiation, myotube size quantification, cytokine production, mitochondrial markers (PGC1α, TFAM), and inflammatory regulators (SPP1, CCL2, CCL7, CCL9, arginase 1). These outcomes will provide detailed insights into how LAMZ influences metabolic and inflammatory pathways in skeletal muscle cells.

10 weeks of LAMZ may have the greatest effect in Example 2, but if five weeks is of greater benefit the treatment timelines may be modified as proposed in Example 3 to five weeks beginning at 30 weeks of age. LAMZ could have a greater impact upon restoring muscle function and reducing the inflammatory response compared to PLX5622 or KP-2326 alone. The depletion of macrophages will likely have an impact upon other immune cell regulation, but it is anticipated that the multiple targets of LAMZ will reduce inflammation (cytokine panel) and restore the cell profile similar to NL, as recognized in flow cytometry. Although there is a link between PTH and inflammation, targeting PTH reduction via KP-2326 will likely not have a similar impact upon the inflammatory response. LAMZ as compared to KP-2326 will likely have a greater proportional difference in reducing macrophages compared to CKD. Depletion of macrophages via PLX5622 is expected to normalize inflammatory outcomes and improve (but not normalize) muscle function due to that lack of a PTH effect. It is feasible that KP combined with PLX could have additive effects, but that is the function of LAMZ, and thus does not warrant this combination. It will be determined if there is an immune-metabolic link though manipulation of macrophages and the impact upon metabolic function and signaling. It may be demonstrated that inflammatory effects are not inextricably linked with metabolism with no metabolic change following immune cell inhibition. Fatty acid PET imaging (¹¹C-palmitate or ¹⁸F-FTO) will be used to examine the effects of immune cell inhibition, PTH, and LAMZ on metabolism. It is likely decreased inflammation from LAMZ will have a greater impact on restoring metabolism compared with KP or PLX treatment. Confirming or refuting an immune-metabolic can provide context for future studies with specific targets. If macrophages can be manipulated with no change in metabolism and a beneficial effect in muscle function, then future directions may focus on anti-inflammatory effects. Regardless, LAMZ could improve both metabolism and inflammation, albeit linked or in parallel. If neither macrophage depletion nor PTH reduction has an effect, fatty acid metabolism will be modulated without exacerbating inflammation via a diet supplement of medium-chain triglycerides. In vitro experiments will identify tyrosine kinase targets that will provide a greater mechanistic understanding of the drug and drug-mediated effects. Further, the in vitro PTH experiments will be used to delineate the role of lowering PTH alone versus a multi-target drug of LAMZ.

### Methods of Example 3

### PET Imaging

PET imaging will be carried out in the Cy/+ and adenine models as previously described for other ligands. Briefly, preclinical PET imaging data will be acquired dynamically over 30-60 minutes. For analysis, 3D volume (VOI) of interest will be manually constructed using a threshold-based segmentation over the skeletal muscle and left ventricle (to estimate the arterial input function, AIF) using Analyze 12 (AnalyzeDirect, Stillwell KS), and dynamic PET time courses extracted across all images. If the tracer exhibits cardiac uptake, the AIF will be generated from the abdominal aorta. Time-activity curves (TACs) will be generated for modeling using three-compartment, five-rate constant tracer kinetic model utilizing eNumerate software package to estimate the model parameters K1, k2, k3, k4, and fractional blood volume (FBV) from TACs generated. Radiometabolite analysis for AIF correction will employ thin-layer chromatography methods derived from venous samples.

### LAMZ/KP/PLX Injections

For LAMZ, animals will be subcutaneously injected with 0.625 mg/kg of LAMZ, 2× daily for 5 and 10 weeks starting at 25 weeks of age. Injection sites are rotated daily at the level of the shoulder (morning) or hip (afternoon) due to the frequency of drug administration to reduce possible irritation. For KP-2326, the preclinical analogue of etelcalcetide, KP-2326 will be intraperitoneally (i.p.) injected (0.6 mg/kg, i.p., 3×/wk) beginning at 25 weeks. For PLX5622, it will be administered via chow at a concentration of 1200 mg/kg, as this dosage has been demonstrated to effectively reduce macrophage populations in previous rodent studies. Flow cytometry will also be used to assess macrophage populations in peripheral blood to further confirm the effectiveness of PLX5622 treatment.

### Functional Assays

Maximal Running Capacity will be performed on a rat treadmill and metabolic testing system will be performed as is typical. Skeletal Muscle Strength and Fatigue will be carried out as is well known and performed as shown in Example 2. Muscle fatigue assessment includes a series of short isometric contractions (less than 500 mS in duration) repeated every 5 seconds, for a duration of three minutes.

### Mitochondria and Metabolic Assays

Mitochondrial outcomes will be monitored by mitochondrial morphology where a small section (<5 mg) of EDL will be fixed with 10% glutaraldehyde and processed for TEM. Quantification of mitochondria will be performed using ImageJ. Mitochondrial outcomes will also be monitored via mitochondrial biogenesis which will be quantitatively measured via proteins of mitochondrial replication and synthesis using the MitoBiogenesis assays (Abcam, Cambridge, MA). Additionally, cellular oxidative capacity will be assessed via citrate synthase activity, which is an index of maximal capacity, using the Citrate Synthase Assay Kit (Sigma-Aldrich, St. Louis, MO). Mitochondrial complex Activity assays for complex I-V activity assays will be performed as well as complex protein expression. Mitochondrial quantity will be assessed via VDAC2. Cytokines will be assessed using commercially available kits per the manufacturer instructions.

### Flow Cytometry

Peripheral blood and tissue samples will be collected from rats and processed to obtain single-cell suspensions. Cells will be stained with a panel of fluorescently labeled antibodies targeting specific immune cell markers to identify and quantify different immune cell populations to determine the effects of LAMZ, KP-2326, and PLX5622 treatment on immune cell populations.

1. A method of improving skeletal health and/or muscle function in a patient experiencing muscle wasting, the method comprising:
administering a therapeutically effective amount of an aminoindazole, or a pharmaceutically acceptable salt thereof, to the patient in need thereof.

2. The method of claim 1, wherein the aminoindazole is locamidazole, or a pharmaceutically acceptable salt thereof, or linifanib, or a pharmaceutically acceptable salt thereof.

3. The method of claim 2, wherein the method improves the handgrip strength, the sit-to-stand test, the 6-minute walk test, the timed up and go test, the gait speed, the isometric knee extensor strength, the short physical performance battery scores, or a combination thereof.

4. The method of claim 2, wherein the improvement of muscle function is muscle strength and/or muscle fatigue.

5. The method of claim 2, wherein the method improves skeletal health by increasing bone cortical thickness and/or porosity.

6. The method of claim 2, wherein the method downregulates inflammatory pathways.

7. The method of claim 6, wherein the method reduces PTH levels.

8. The method of claim 2, wherein the method normalizes macrophage expression.

9. The method of claim 2, wherein the method reduces macrophage infiltration in skeletal muscle.

10. The method of claim 2, wherein the method upregulates a gene in a metabolic pathways, improves fatty acid uptake and esterification, or both.

11. The method of claim 10, wherein the metabolic pathway is selected from the group consisting of fatty acid oxidation, ATP synthesis, and the electron transport chain.

12. The method of claim 2, wherein the method improves mitochondrial morphology.

13. The method of claim 12, wherein the improved mitochondrial morphology comprises reduced mitochondrial swelling and/or reduced disorganization of mitochondrial cristae.

14. The method of claim 2, wherein mitochondria impairment is decreased when compared to a patient not receiving the aminoindazole.

15. The method of claim 2, wherein the aminoindazole is locamidazole, or a pharmaceutically acceptable salt thereof.

16. A method of improving skeletal health and/or muscle strength in a patient having chronic kidney disease, the method comprising:
administering a therapeutically effective amount of an aminoindazole, or a pharmaceutically acceptable salt thereof, to the patient in need thereof.

17. The method of claim 16, wherein the aminoindazole is locamidazole, or a pharmaceutically acceptable salt thereof, or linifanib, or a pharmaceutically acceptable salt thereof.

18. The method of claim 17, wherein the aminoindazole is administered in combination with an additional therapeutic.

19. The method of claim 17, wherein the additional therapeutic is a GLP-1 inhibitor, or an anti-inflammatory drug.

20. The method of claim 16, wherein the patient has an amyotrophic disease.
